(19) United States
(12) Patent Application Publication
(10) Pub. No.: US 2023/0126296 A1
(43) Pub. Date: Apr. 27, 2023

(54) SYSTEMS AND METHODS FOR GUIDED INTERVENTION

(71) Applicants: Massachusetts Institute of Technology, Cambridge, MA (US); The General Hospital Corporation, Boston, MA (US)

(72) Inventors: Laura J. Brattain, Lexington, MA (US); Anthony E. Samir, Newton, MA (US); Brian A. Telfer, Lincoln, MA (US); Matthew Johnson, Medford, MA (US); Nancy DeLosa, Westford, MA (US); Lars Gjesteby, Maynard, MA (US); Theodore T. Pierce, Auburndale, MA (US); Joshua Werblin, Boston, MA (US)

(21) Appl. No.: 17/971,073

(22) Filed: Oct. 21, 2022

Related U.S. Application Data
(60) Provisional application No. 63/270,376, filed on Oct. 21, 2021.

(51) Int. Cl. A61B 8/08; A61B 8/12

(52) U.S. Cl. CPC A61B 8/08; A61B 8/12; A61B 8/0841

(57) ABSTRACT

Systems and methods are provided for semi-automated, portable, ultrasound guided cannulation. The systems and methods provide for image analysis to provide for segmentation of vessels of interest from image data. The image analysis provides for guidance for insertion of a cannulation system into a subject which may be accomplished by a non-expert based upon the guidance provided. The guidance may include an indicator or a mechanical guide to guide a user for inserting the vascular cannulation system into a subject to penetrate the vessel of interest.

### CROSS-REFERENCE TO RELATED APPLICATIONS

This application is based on, claims priority to, and incorporates herein by reference, U.S. Provisional Application Ser. No. 63/270,376, filed Oct. 21, 2021, and entitled “SYSTEMS AND METHODS FOR PORTABLE ULTRASOUND GUIDED CANNULATION.”

### STATEMENT REGARDING FEDERALLY SPONSORED RESEARCH

This invention was made with government support under FA8702-15-D-0001 awarded by the U.S. Army and Defense Health Agency. The government has certain rights in the invention.

### BACKGROUND

Insertion of catheters into blood vessels, veins, or arteries can be a difficult task for non-experts or in trauma applications because the vein or artery may be located deep within the body, may be difficult to access in a particular patient, or may be obscured by trauma in the surrounding region to the vessel. Multiple attempts at penetration may result in extreme discomfort to the patient, loss of valuable time during emergency situations, or in further trauma. Furthermore, central veins and arteries are often in close proximity to each other. While attempting to access the internal jugular vein, for example, the carotid artery may instead be punctured, resulting in severe complications or even mortality due to consequent blood loss due to the high pressure of the blood flowing in the artery. Associated nerve pathways may also be found in close proximity to a vessel, such as the femoral nerve located nearby the femoral artery, puncture of which may cause significant pain or loss of function for a patient.

To prevent complications during cannulation, ultrasonic instruments can be used to determine the location and direction of the vessel to be penetrated. One method for such ultrasound guided cannulation involves a human expert who manually interprets ultrasound imagery and inserts a needle. Such a manual procedure works well only for experts who perform the procedure regularly so that they may accurately cannulate a vessel.

Systems have been developed in an attempt to remove or mitigate the burden on the expert, such as robotic systems that use a robotic arm to insert a needle. These table-top systems and robotic arms are too large for portable use, such that they may not be implemented by medics at a point of injury. In addition, previous systems have been limited to peripheral venous access, may not be used to cannulate more challenging vessels or veins, and may not provide a sufficient level of accuracy to reliably place a needle into a desired vessel.

Still other systems have been used to display an image overlay on the skin to indicate where a vessel may be located, or otherwise highlight where the peripheral vein is located just below the surface. However, in the same manner as above, these systems are limited to peripheral veins, and provide no depth information that may be used by a non-expert to guide cannulation, not to mention failures or challenges associated with improper registration.

Therefore, there is a need for techniques for improved cannulation of blood vessels that is less cumbersome, more accurate, and able to be deployed by a non-expert.

### SUMMARY OF THE DISCLOSURE

The present disclosure addresses the aforementioned drawbacks by providing systems and methods for guided vascular cannulation with increased accuracy. The systems and methods provide for image analysis to provide for segmentation of vessels of interest from image data. The image analysis provides guidance for insertion of a cannulation system into a subject and may be accomplished by a non-expert based upon the guidance provided. The guidance may include an indicator or a mechanical guide to guide a user when inserting the vascular cannulation system into a subject to penetrate the vessel of interest.

In one configuration, a system is provided for guiding an interventional device in an interventional procedure of a subject. The system includes an ultrasound probe, a guide system coupled to the ultrasound probe and configured to guide the interventional device into a field of view (FOV) of the ultrasound probe, a non-transitory memory having instructions stored thereon, and a processor configured to access the non-transitory memory and execute the instructions. The processor is caused to access image data acquired from the subject using the ultrasound probe. The image data include at least one image of a target structure of the subject. The processor is also caused to determine, from the image data, a location of the target structure within the subject and determine an overshoot estimation for the interventional device based upon the location of the target structure and guide the interventional device to penetrate the target structure without penetrating a distal wall of the target structure based upon the overshoot estimation.

In another configuration, a system is provided for guiding an interventional device in an interventional procedure of a subject. The system includes an ultrasound probe, a guide system coupled to the ultrasound probe and configured to guide the interventional device into a field of view (FOV) of the ultrasound probe, a non-transitory memory having instructions stored thereon, and a processor configured to access the non-transitory memory and execute the instructions. The processor is caused to access image data acquired from the subject using the ultrasound probe. The image data include at least one image of a target structure of the subject. The processor is also caused to determine, from the image data, a cross section of the target structure within the subject. The processor is also caused to fit an ellipse for the cross section of the target structure to determine a centroid for the target structure and guide the interventional device to the centroid to penetrate the target structure.

The foregoing and other aspects and advantages of the present disclosure will appear from the following description. In the description, reference is made to the accompanying drawings that form a part hereof, and in which there is shown by way of illustration a preferred embodiment. This embodiment does not necessarily represent the full scope of the invention, however, and reference is therefore made to the claims and herein for interpreting the scope of the invention. Like reference numerals will be used to refer to like parts from Figure to Figure in the following description.

### BRIEF DESCRIPTION OF THE DRAWINGS

FIG. 1 is a schematic diagram of a non-limiting example ultrasound system that can implement the systems and methods described in the present disclosure.

FIG. 2 is a schematic diagram of a non-limiting example configuration for guiding needle insertion into a vessel of interest using an ultrasound probe.

FIG. 3A is a flowchart of non-limiting example steps for a method of operating a system for guiding vascular cannulation.

FIG. 3B is a graph of a non-limiting example of a blood flashback method for confirming placement within a vessel.

FIG. 3C is a graph of a non-limiting example for dynamic speed control of a needle for penetrating a vessel.

FIG. 3D is graph of a non-limiting example of force feedback for controlling a needle for penetration of a vessel.

FIG. 4A is a flowchart of non-limiting example steps for a method of fitting an ellipse for determining a vessel centroid.

FIG. 4B is a flowchart of non-limiting example steps for a method of guiding needle penetration of a vessel of interest.

FIG. 4C is a flowchart for a non-limiting example automatic gain control.

FIG. 5 is a block diagram of an example system that can implement a vessel of interest image processing system for generating images of a vessel of interest or otherwise measuring or predicting a location for a vessel of interest using a hybrid machine learning and mechanistic model.

FIG. 6 is a block diagram of example hardware components of the system of FIG. 5.

FIG. 7A is a perspective view of a non-limiting example interventional device guide coupled to an ultrasound probe.

FIG. 7B is a side view of the interventional device guide of FIG. 7A.

FIG. 7C is a side view of the base and ultrasound probe fixture for the interventional device guide of FIG. 7B.

FIG. 7D is a cross-section of a non-limiting example cartridge compatible with the injection assemble of FIG. 7B.

FIG. 8A is a perspective view of a non-limiting example interventional device guide integrated with an ultrasound probe.

FIG. 8B is an exploded view of the integrated interventional device guide and ultrasound probe of FIG. 8A.

FIG. 9 is perspective view of a non-limiting example cricothyrotomy cartridge for use in accordance with the present disclosure.

FIG. 10A is a side view of inserting a non-limiting example dilating component into the interventional device guide.

FIG. 10B is a side view of aligning the non-limiting example dilating component with the interventional device guide and advancing a needle to guide the non-limiting example dilating component into the subject.

FIG. 10C is a side view of advancing the non-limiting example dilating component over the needle and into the subject.

FIG. 10D is a side view of retracting the needle and leaving the non-limiting example dilating component in the subject.

FIG. 10E is a side view of removing the interventional device guide and leaving the non-limiting example dilating component in the subject.

### DETAILED DESCRIPTION

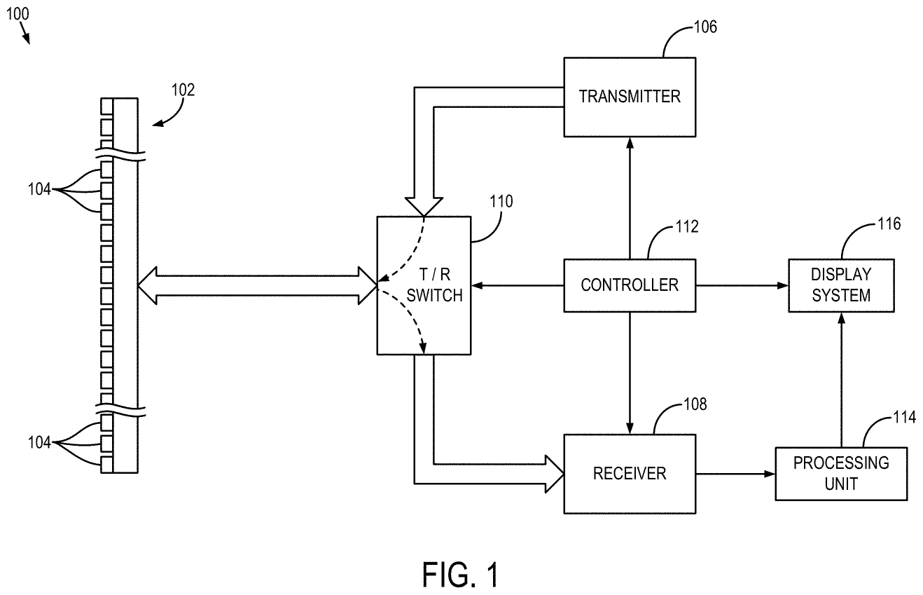

FIG. 1 illustrates an example of an ultrasound system 100 that can implement the methods described in the present disclosure. The ultrasound system 100 includes a transducer array 102 that includes a plurality of separately driven transducer elements 104. The transducer array 102 can include any suitable ultrasound transducer array, including linear arrays, curved arrays, phased arrays, and so on. Similarly, the transducer array 102 can include a 1 D transducer, a 1.5 D transducer, a 1.75 D transducer, a 2 D transducer, a 3 D transducer, and so on.

When energized by a transmitter 106, a given transducer element 104 produces a burst of ultrasonic energy. The ultrasonic energy reflected back to the transducer array 102 (e.g., an echo) from the object or subject under study is converted to an electrical signal (e.g., an echo signal) by each transducer element 104 and can be applied separately to a receiver 108 through a set of switches 110. The transmitter 106, receiver 108, and switches 110 are operated under the control of a controller 112, which may include one or more processors. As one example, the controller 112 can include a computer system.

The transmitter 106 can be programmed to transmit unfocused or focused ultrasound waves. In some configurations, the transmitter 106 can also be programmed to transmit diverged waves, spherical waves, cylindrical waves, plane waves, or combinations thereof. Furthermore, the transmitter 106 can be programmed to transmit spatially or temporally encoded pulses.

The receiver 108 can be programmed to implement a suitable detection sequence for the imaging task at hand. In some embodiments, the detection sequence can include one or more of line-by-line scanning, compounding plane wave imaging, synthetic aperture imaging, and compounding diverging beam imaging.

In some configurations, the transmitter 106 and the receiver 108 can be programmed to implement a high frame rate. For instance, a frame rate associated with an acquisition pulse repetition frequency (“PRF”) of at least 100 Hz can be implemented. In some configurations, the ultrasound system 100 can sample and store at least one hundred ensembles of echo signals in the temporal direction.

The controller 112 can be programmed to implement an imaging sequence using the techniques described in the present disclosure, or as otherwise known in the art. In some embodiments, the controller 112 receives user inputs defining various factors used in the design of the imaging sequence.

A scan can be performed by setting the switches 110 to their transmit position, thereby directing the transmitter 106 to be turned on momentarily to energize transducer elements 104 during a single transmission event according to the implemented imaging sequence. The switches 110 can then be set to their receive position and the subsequent echo signals produced by the transducer elements 104 in response to one or more detected echoes are measured and applied to the receiver 108. The separate echo signals from the transducer elements 104 can be combined in the receiver 108 to produce a single echo signal.

The echo signals are communicated to a processing unit 114, which may be implemented by a hardware processor and memory, to process echo signals or images generated from echo signals. As an example, the processing unit 114 can guide cannulation of a vessel of interest using the methods described in the present disclosure. Images produced from the echo signals by the processing unit 114 can be displayed on a display system 116.

In some configurations, a non-limiting example method may be deployed on an imaging system, such as a commercially available imaging system, to provide for a portable ultrasound system with vessel cannulation guidance. The method may locate a vessel of interest, such as a vein or an artery as a user or medic moves an ultrasound probe. The system and method may provide real-time guidance to the user to position the ultrasound probe to the optimal needle insertion point. The probe may include one or more of a fixed needle guide device, an adjustable mechanical needle guide, a displayed-image needle guide, and the like. An adjustable guide may include adjustable angle and/or depth. The system may guide, or communicate placement or adjustments for the guide for the needle. The system may also regulate the needle insertion distance based upon the depth computed for the vessel of interest. The user may then insert a needle through the mechanical guide attached to the probe or displayed guide projected from the probe in order to ensure proper insertion. During needle insertion, the system may proceed to track the target blood vessel and the needle until the vessel is penetrated. A graphical user interface may be used to allow the medic to specify the desired blood vessel and to provide feedback to the medic throughout the process.

For the purposes of this disclosure and accompanying claims, the term “real time” or related terms are used to refer to and defined a real-time performance of a system, which is understood as performance that is subject to operational deadlines from a given event to a system's response to that event. For example, a real-time extraction of data and/or displaying of such data based on acquired ultrasound data may be one triggered and/or executed simultaneously with and without interruption of a signal-acquisition procedure.

In some configurations, the system may automate all ultrasound image interpretation and insertion computations, while a medic or a user may implement steps that require dexterity, such as moving the probe and inserting the needle. Division of labor in this manner may avoid using a dexterous robot arm and may result in a small system that incorporates any needed medical expertise.

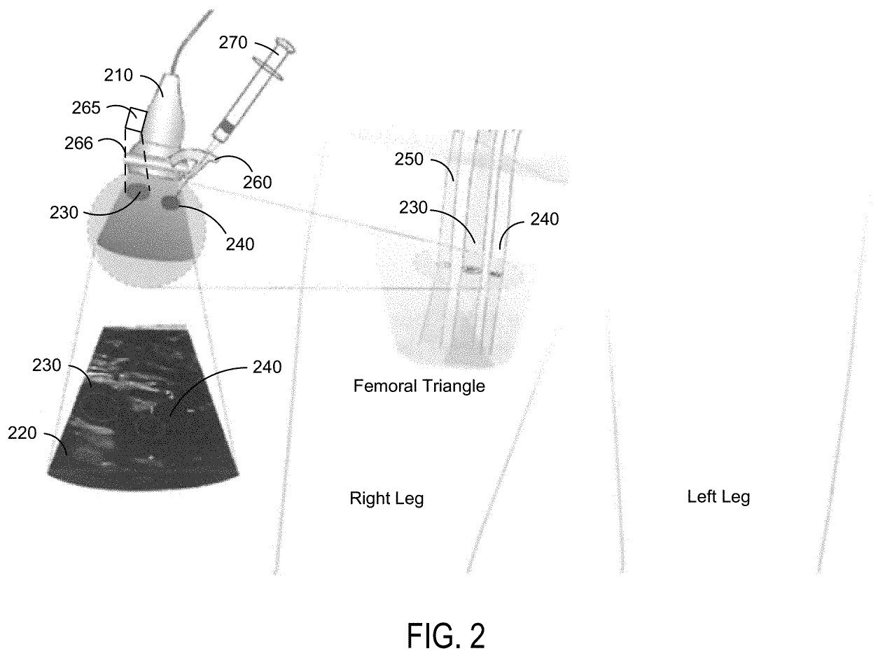

Referring to FIG. 2, a diagram is shown depicting a non-limiting example embodiment for guiding needle insertion into a femoral artery 230 or femoral vein 240. An ultrasound probe 210 is used to acquire an image 220 of a region of interest that includes a portion of the femoral artery 230, femoral vein 240 and other objects of interest such as femoral nerve 250. The locations of the femoral artery 230, femoral vein 240, and femoral nerve 250 may be annotated on the image 220. A mechanical needle guide 260 may be included to guide a needle 270 to penetrate the vessel of interest, such as femoral vein 240 as shown. In some configurations, visual needle guide 265 may be included where a penetration guide image 266 is projected onto the surface of a subject to guide a needle 270 to penetrate the vessel of interest, such as to femoral artery 230 as shown. Penetration guide image 266 may reflect the actual size or depth of the vessel of interest for penetration when projected onto the subject, or may provide other indicators such as measurements or a point target for penetration, and the like.

The vessels of interest may include a femoral artery, femoral vein, jugular vein, peripheral veins, subclavian vein, and or other vessels or non-vessel structures. Non-limiting example applications may include aiding a medic in performing additional emergency needle insertion procedures, such as needle decompression for tension pneumothorax (collapsed lung) and needle cricothyrotomy (to provide airway access). Portable ultrasound may be used to detect tension pneumothorax and needle insertion point (in an intercostal space, between ribs) or to detect the cricothyroid membrane and needle insertion point.

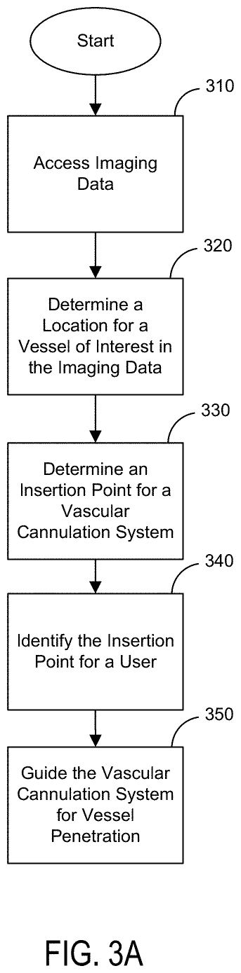

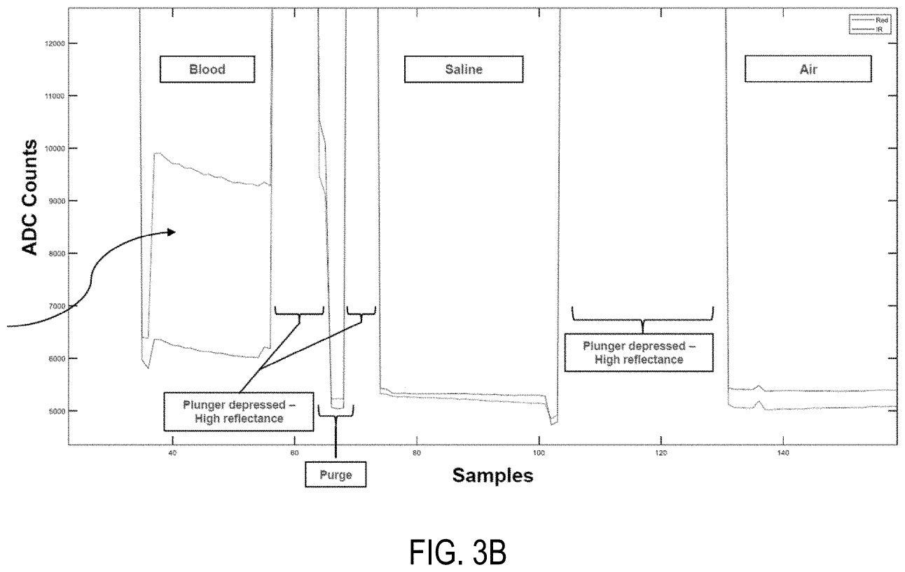

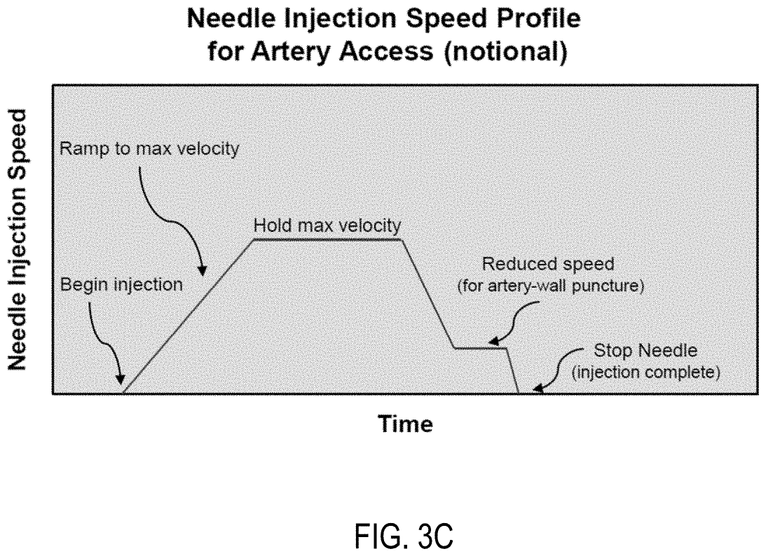

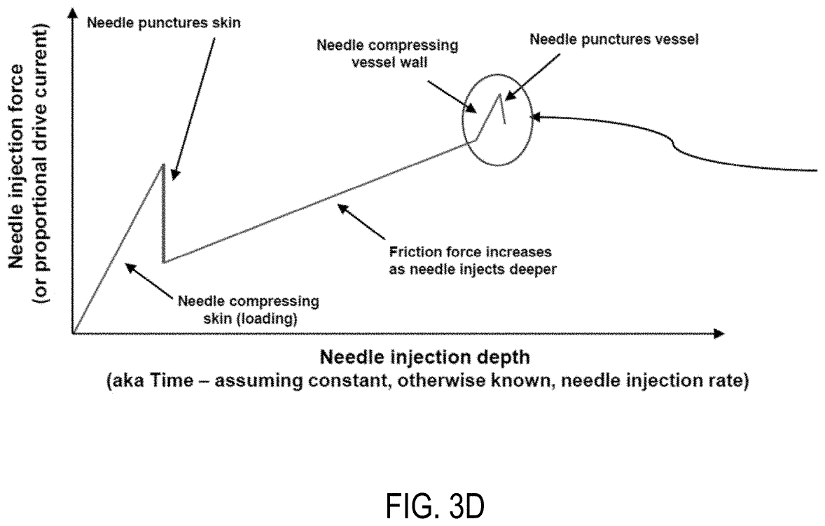

Referring to FIG. 3A, a non-limiting example steps of a method of operating a system for guiding vascular cannulation is shown. At step 310, imaging data is accessed. This may be achieved by performing an imaging acquisition and/or accessing pre-acquired image data. Imaging data may include ultrasound data, and/or may include any other form of medical imaging data, such as magnetic resonance imaging (MRI), computed tomography (CT), PET, SPECT, fluoroscopy, and the like. Using the imaging data, a vessel of interest may be determined at step 320. The location may be determined by segmenting the vessels of interest in the imaging data. Vessels of interest may include a femoral artery, femoral vein, jugular vein, peripheral veins, subclavian vein, and the like. An insertion point may then be determined at step 330 for a vascular cannulation system. Determining the insertion point may be based upon the determined location for the vessel of interest and calculating a depth and a pathway for the cannulation system from the surface of a subject to the vessel of interest without the cannulation system penetrating other organs of interest, such as a nerve. The insertion point may be determined for a user at step 340. The insertion point may be identified by illuminating a portion of the surface of a subject, or by adjusting a mechanical needle guide to the appropriate settings for the user, and the like. Depth of the needle penetration may also be controlled by a setting or a height of the mechanical guide. The vascular cannulation system may be guided to the vessel of interest for vessel penetration at step 350. Guiding the vascular cannulation system may include acquiring images of the vessel of interest and the vascular cannulation system as the cannulation system is inserted into the subject and displaying the tracked images for the user.

Any ultrasound probe may be used in accordance with the present disclosure, including 1D, 2D, linear, phased array, and the like. In some configurations, an image is displayed for a user of the vessel of interest with any tracking information for the needle overlaid on the image. In some configurations, no image is displayed for a user and instead only the insertion point may be identified by illuminating a portion of the surface of a subject. In some configurations, no image is displayed and the user is only informed of the probe reaching the proper location whereby a mechanical needle guide is automatically adjusted to the appropriate settings, such as angle and/or depth to target a vessel of interest. The user may be informed of the probe reaching the proper location by any appropriate means, such as light indicator, a vibration of the probe, and the like.

In some configurations, identification of placement of the ultrasound transducer at a target location may be performed automatically by the system. Image data may be used for identifying anatomy, such as a femoral triangle, jugular region, and the like, and may be accessed by the system to provide automatic identification for where the ultrasound transducer has been placed. In some configurations, a user may specify the vessel of interest to be targeted, such as whether to target an artery or a vein. In a non-limiting example combination of the configurations, the location of the ultrasound transducer on the subject may be automatically determined along with the anatomy being imaged, with the user specifying the vessel of interest to target in the automatically identified anatomy. A minimum of user input may be used in order to mitigate the time burden on a user.

Segmenting the vessels of interest may be based on machine learning of morphological and spatial information in the ultrasound images. In some configurations, a neural network may be deployed for machine learning and may learn features at multiple spatial and temporal scales. Vessels of interest may be distinguished based on shape and/or appearance of the vessel wall, shape and/or appearance of surrounding tissues, and the like. In a non-limiting example, stiffer walls and a circular shape may be used to distinguish an artery in an image, whereas an ellipsoidal shape may be used to identify a vein. Real-time vessel segmentation may be enabled by a temporally trained routine without a need for conventional post-hoc processing.

Temporal information may be used with segmenting the vessels of interest. Vessel appearances and shape may change with movement of the anatomy over time, such as changes with heartbeat, or differences in appearance between hypotensive and normal-tensile situations. Machine learning routines may be trained with data from multiple time periods with differences in anatomy being reflected over the different periods of time. With a temporally trained machine learning routine, vessel segmentation may be performed in a robust manner over time for a subject without misclassification and without a need to find a specific time frame or a specific probe position to identify vessels of interest.

In some configurations, to prevent any potential misclassifications conflicting information checks may be included in the system. A conflicting information check may include taking into consideration the general configuration of the anatomy at the location of the probe. In a non-limiting example, if the system initially identifies two arteries at a location of the probe, but the general anatomy at the location of the probe indicates that an artery and a vein should be returned as results instead, then the system will automatically correct to properly identify an artery and a vein instead of the mistaken two arteries to prevent a misclassification.

Identifying an insertion point for a user may also include where the system automatically takes into account the orientation of the probe on a body. A conventional ultrasound probe includes markings on the probe to indicate the right vs left side of probe, which allows a user to orient a probe such that the mark is on the right of the patient, for example. The probe orientation may be also be determined from an analysis of the acquired ultrasound images, or monitoring of the orientation of the markings, such as by an external camera. In some configurations, the needle guide attachment may be configured fit into the markings on the probe to ensure that the device is consistent with the orientation of the probe.

In some configurations, a vibrating needle tip may be used to promote vessel penetration. A vibrating needle tip may also be used to address vessel wall tenting. Vessel wall tenting is a form of vessel wall deformation due to the pressure of a needle that takes place prior to a needle puncturing the vessel. Insertion through a relatively robust sidewall of an artery may present challenges due to lateral displacement of the vessel relative to a needle tip resulting from contact between the two, such as vessel wall tenting. Needle tip vibration may be used to more easily puncture a vessel wall, such as an artery, by reducing the amount of pressure needed to puncture the vessel and thereby may also reduce the amount of vessel wall tenting. Reducing the amount of insertion force may also allow for a reduction in the size of the drive motor used to insert the needle. The vibration of the needle tip may be tuned in frequency, magnitude, or timing, and the like, to be optimized for arterial and/or vein insertion. Needle tip vibration may also reduce the likelihood of artery dissection, misses, or tears from “glancing shots” near the vessel.

A vibrating needle tip may include vibration frequencies that are adjusted or changed with depth or needle length in order to maintain a vibration at resonance in the needle. As the length of the needle increases, or the depth of the needle in the subject increases, the frequency of the vibration may be reduced to maintain a resonance frequency in the needle. In some configurations, the frequencies used may be around 100 Hz up to and including 1000 Hz. In some configurations, several hundred Hertz may be used for a frequency. In a non-limiting example, 300 Hz is used for the needle tip vibration frequency.

In some configurations, an estimation of needle overshoot may be used in order to provide for higher accuracy in delivering the needle into the desired vessel, and to ensure a greater depth control for needle delivery. Vessel tenting may also be addressed with a safe needle overshoot estimation. Needle overshoot may be estimated as a function of vessel depth and distance to a posterior wall of the vessel, such as indicated in non-limiting example eqs. (1) and (2):

$$Insertion angle ( θ° ) = - 0.0145 * {D}^{2} + 1.7338 * D + 15.445 \qquad ( 1 )$$

$$Overshoot ( h ) = [ \frac{y}{sin ( θ )} ] - 1 mm \qquad ( 2 )$$

Where y represents the distance to the posterior wall of the vessel, h the overshoot estimation, D the depth of the centroid of the vessel, and θ the insertion angle of the needle.

Needle overshoot estimation may be used to facilitate successful cannulation and is accomplished by establishing overshoot limits that determine how much deeper than the targeting centroid the needle tip may be allowed to extend. In non-limiting examples, a calculated overshoot may be based on the location of a critical structure or the location of a vessel wall that is deeper than a targeted centroid. For example, in some configurations, the needle may stop 1 mm short, 3 mm short, or 7 mm short of a critical structure or vessel wall that is deep to the centroid, or a length as determined by the depth, size, and/or diameter of the vessel or the needle. After the needle overshoots beyond the targeting centroid, the needle tip may be retracted to the centroid after an initial overshoot. The needle may also retract to or within a desired distance, such as, for example, 1 mm of the anterior vessel wall before returning to the vessel centroid or advancing to a new setpoint, for example, 1 mm beyond the posterior vessel wall. Furthermore, in a non-limiting example, an absolute lower limit may be set, for example, 3 mm, for needle overshoot, such as when the calculated value of overshoot is less than that which would be expected to provide increased likelihood of successful vessel penetration. Similarly, an absolute maximum limit of needle overshoot may be set when the calculated value of overshoot is greater than that which would be expected to provide increased likelihood of successful vessel penetration while increasing risk that a non-target structure is damaged. In some non-limiting examples, this maximum limit, if used, may be 7 mm.

In some configurations after needle injection, a blood flashback method may be used to confirm the needle has penetrated a vessel. A syringe or other hollow structure may be connected to the proximal end of the needle, and the plunger may be pulled back to create suction. If blood is pulled into the hollow chamber it is determined that the needle tip is in the blood vessel. An automated assessment of blood flashback may be used to determine if a needle has been placed in a vessel, such as when using a motor driven system for needle insertion.

Referring to FIG. 3B, a graph of a non-limiting example blood flashback method is shown. The optical signature of blood may be used in an automated system using blood flashback to determine if blood is present after the needle has penetrated a vessel. The optical signature of blood is unique from that of water or air. In a non-limiting example, a light source such as an LED may have a wavelength of 532 nm that may be used to illuminate a blood sample to determine if blood is present as blood absorbs light approximately 5 orders of magnitude stronger than water at this wavelength. Other wavelengths may be used, or a plurality of wavelengths may be used, such as in a blood oximetry system that may be used in addition to the blood flashback method. In a non-limiting example, a green LED of 537 nm may be used with a red LED of 660 nm, and an infrared LED of 880 nm. Multiple wavelengths may provide for a more robust determination of blood flashback and/or to determine blood oxygenation percentage, such as by a ratio of received light. Blood oxygenation may also be used for distinguishing between arteries or veins for diagnostic purposes or for confirmation of the target vessel. Contrast is strong across a wide range of wavelengths such that a sensor could employ a light source, such as an LED, across a range of wavelengths. In a non-limiting example, a light source may include a broadband light source. In a non-limiting example, light sources with a 1.0-1.2 μm separation may be used in parallel to demonstrate that the optical path is not simply blocked.

In some configurations, a blood flashback method may use blood as a liquid shutter in an optical system where a needle is advanced towards a target vessel until blood flashback is detected. Once blood is detected, the needle has been determined to have penetrated the vessel and the needle may be stopped. An indicator may be used to inform a user on the status of the needle, such as by using a green LED in a non-limiting example to convey that the needle insertion has begun. A photodiode may be used to receive light and produce a proportional current that may be translated into a voltage and read into a microcontroller. A successful injection may be determined when the photodiode current output drops to a level consistent with a low level of light received from the indicator or green LED.

In some configurations, the blood flashback method may include using the difference in optical reflection and/or optical index at various wavelengths. A multiple wavelengths approach may be more robust to make a blood/no-blood determination and to quantify blood oxygenation. An optical reflection approach may be easier to integrate into a system as the transmit/receive apertures may be more nearly co-located. Blood oxygenation data can also provide insight into which vessel was punctured and other info related to patient health.

Referring to FIG. 3C, a non-limiting example of dynamic needle speed is shown. In some configurations, dynamic needle speed may be used to promote vessel penetration. Dynamic needle speed may minimize the amount the needle tip may slide off the side of the vessel by reducing the needle speed as the tip nears the vessel wall. Reasons for a needle missing the intended vessel include an inability to puncture the vessel wall, such as due to tenting, and improper effective injection length due to operator or patient motion. By ramping the needle to maximum velocity after injection, then reducing speed as the needle approaches the vessel, and stopping the needle once injection is complete, the vessel may be penetrated more easily, vessel tenting may be mitigated, and accuracy may be improved.

Referring to FIG. 3D, a non-limiting example of force feedback for controlling a needle is shown. Force feedback may be used from a vessel puncture event where the feedback is intended to detect a “popping” feeling an operators may sense as the needle punctures the vessel wall. A force sensor in line with needle or drive mechanism may be used to provide the feedback. A monitor for the current level of needle drive motor may also be used to provide the feedback.

In some configurations, determination of a vessel centroid may be used to improve vessel targeting accuracy for penetration. Vessel ellipse fitting may be used to accurately localize a vessel centroid and/or vessel walls. Ultrasound image data may be accessed or acquired that includes a cross section of the target vessel for ellipse fitting. A bounding box (Bbox) may be extracted that selects the vessel cross section within an ultrasound image. An Otsu threshold may be used to determine the general outline for a vessel. The vessel general outline may be eroded until nearly connected and dilation may be used to expand the eroded boundary out to the vessel walls. A contour fitting algorithm may be used to segment the lumen walls in the true shape of the vessel. Using the detected bounding box center as a seed point, spokes may be generated at desired intervals, such as at 10-degree intervals, and extended until an intensity difference threshold is reached, indicating the tissue wall. The spokes may be filtered to remove any that project past the true vessel wall. The endpoints of all valid spokes may then be used to calculate a best-fit ellipse. The ellipse center may be computed as an estimate of the vessel centroid, which is intended to improve the needle insertion guidance. The major and minor axes of the ellipse can also provide insight on a patient's hemodynamic status (e.g. vasoconstriction).

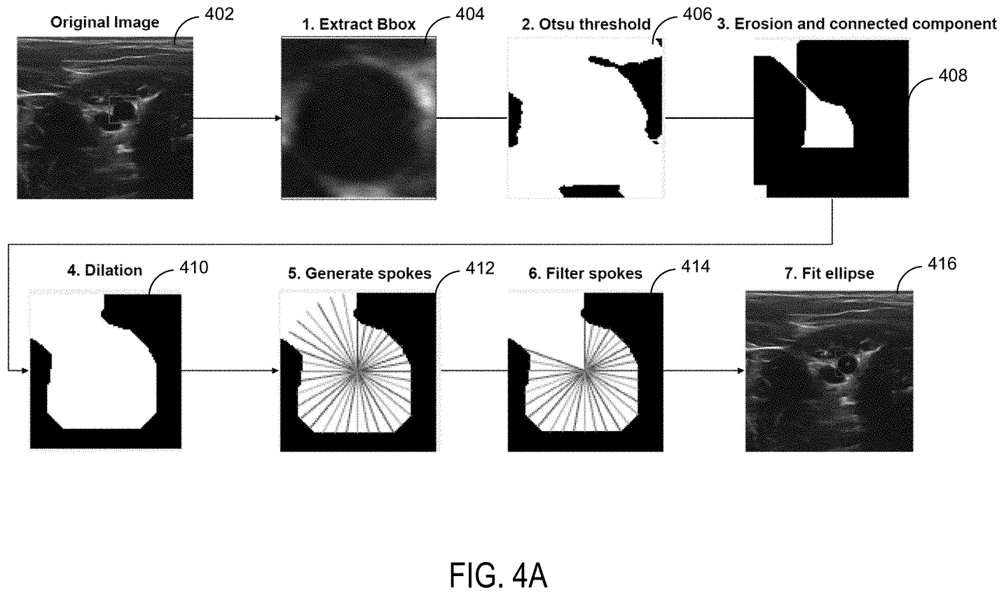

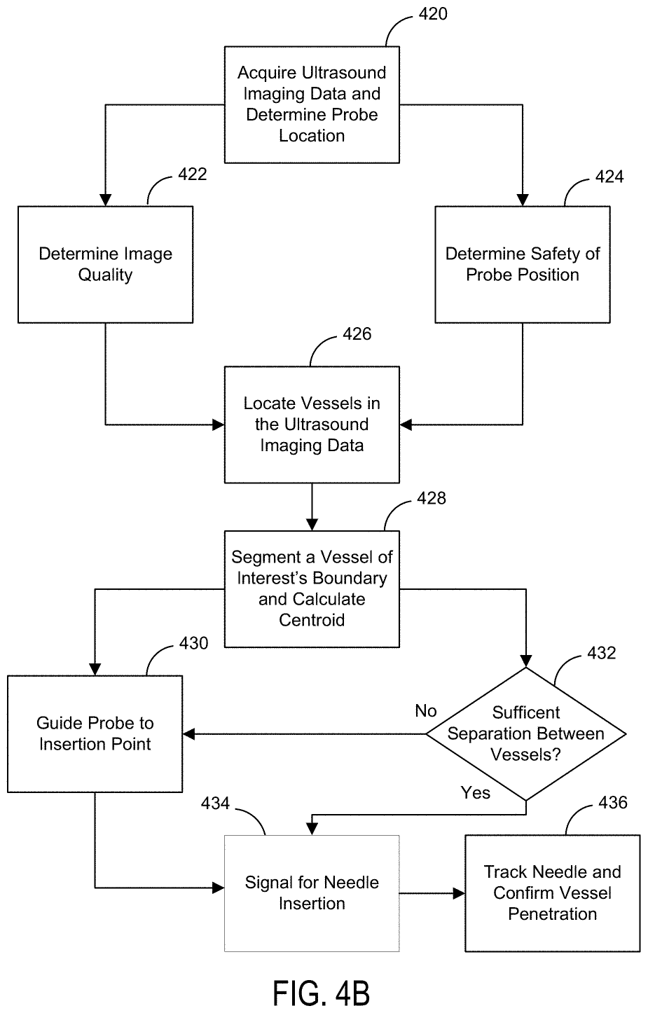

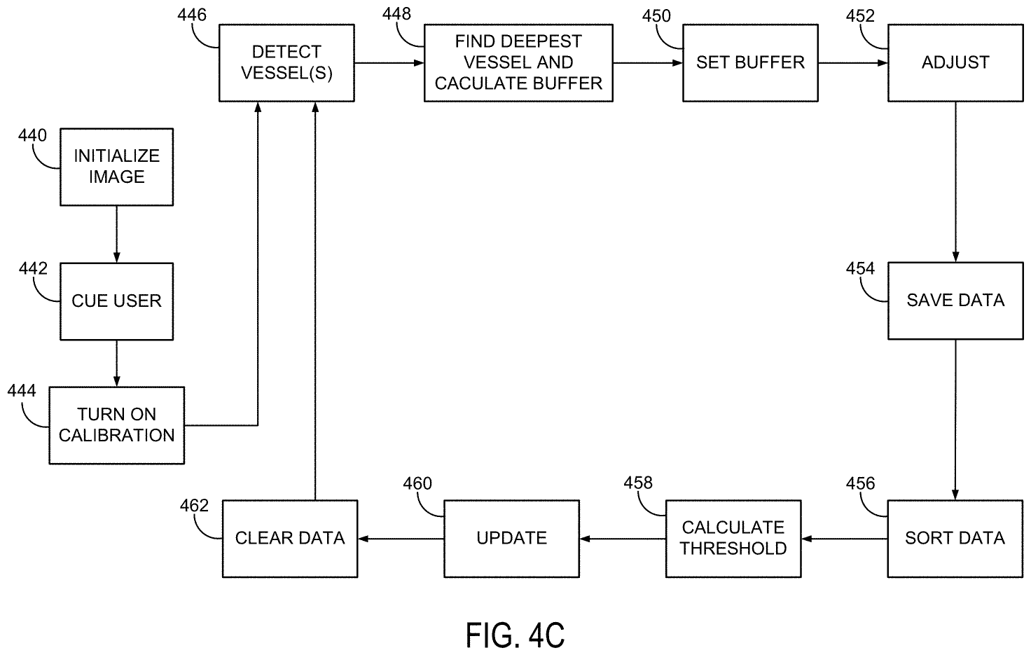

Referring to FIG. 4A, non-limiting example steps are shown in a flowchart for ellipse fitting algorithm steps on an example artery detection. First, a full image is produced at step 402 and the vessel bounding box is extracted at step 404 from the full image from step 402. Then, Otsu thresholding is performed at step 406 on the bounding box to create a binary map separating vessel lumen from surrounding tissue. Erosion and connected components analysis is applied at step 408 to the binary image to isolate pixels associated with the target vessel lumen. The erosion may be performed with an adaptive kernel size proportional to 25% of the vessel height or width (whichever measurement is smaller). Then, an image dilation step 410 restores the target vessel lumen to its original size while omitting most of the surrounding tissue. The dilation may be performed with an adaptive kernel size proportional to 22% of the vessel height or width (whichever measurement is smaller). Lines are then generated at step 412 from the vessel centroid in a spoke pattern in the binary image. The spokes are grown until the boundary between the binary pixel value changes from 1 to 0 or the edge of the binary image is reached. All spokes whose lengths are within 1.5 standard deviations of the mean spoke length are retained at step 414, and an ellipse is fit to the endpoints of these remaining spokes at step 416.

Dynamic vessel centroid targeting may be used based on the diameter of the vessel and a safety check may also be performed as part of needle insertion. A safety check may include confirming that there are no critical structures, such as a bone, an unintended blood vessel, a non-target organ, a nerve, or other structure that should be avoided, intervening on the needle's path to penetrate the vessel. The safety check may also include forcing the system to change the location of the penetration to avoid penetrating such critical structures. In some configurations, the safety check may include confirming the needle has penetrated the vessel of interest by the tracking and guidance. The safety check may also include determining that the user is holding the system in a stable position, by verifying from the ultrasound image or from an inertial measurement unit on the handle of the system. While the safety check may prevent needle insertion within a certain distance of a critical structure, the dynamic vessel centroid targeting may expand the range of available safe insertion angles/positions as a needle may be permitted to deviate from targeting the centroid of the vessel to instead be able to target a space between the centroid and the vessel wall.

Referring to FIG. 4B, non-limiting example steps are shown in a flowchart setting forth a method of guiding needle penetration of a vessel of interest. Ultrasound imaging data is acquired and a probe location is determined at step 420. An image quality may be determined at step 422, and the safety of the probe location for penetrating a vessel in the subject may be determined at step 424. Vessels may be located in the imaging data at step 426. A vessel of interest's boundary may be segmented and a centroid calculated for the vessel of interest at step 428. The probe may be guided to an insertion point at step 430. Sufficient separation between vessels may be determined or confirmed at step 432. If there is not sufficient separation, the probe may be guided to a new insertion position at step 430. If there is sufficient separation, then a signal may be provided to a user to proceed with needle insertion at step 434. Such a signal may be provided on a graphical user interface, or a light in the probe, and the like. The needle may be tracked and vessel penetration confirmed at step 436.

In some configurations, the method includes guiding a user in placement of the ultrasound probe on the subject. A target for penetration may be identified, such as by machine learning in accordance with the present disclosure, and localized. A user may then be guided in which direction to move the ultrasound probe for placement over an identified target. Once the ultrasound probe has reached the target location, a signal may indicate for the user to stop moving the probe. Guidance may be provided by the signal, such as the light on the probe, in a non-limiting example. Needle placement and penetration may proceed after the location of the target has been reached.

In some configurations, vessel branching may be used to guide needle insertion. If vessel branching is detected, the system may indicate to the user to move the device away from that location so as to avoid penetrating a branched vessel. Vessel branching/bifurcation is defined as the point where the deep femoral artery bifurcates from the common femoral artery (CFA) and the femoral vein bifurcates from the common femoral vein. Images of this region may be collected, and labeled as a special class for machine learning or AI algorithm training to provide automated guidance to a user on avoiding vessel branching. The CFA bifurcation is a mean of 7.5 cm below the inguinal ligament, so this landmark may be used as a lower bound and the system may instruct the user to move cranially until the bifurcation is no longer detected before an injection can occur.

Referring to FIG. 4C, a flowchart setting forth a non-limiting example of a process for automatic gain control. The process may start by initializing an image at step 440. In this non-limiting example, gain for the ultrasound system may be automatically controlled based on depth. In this case, image initialization may be performed for a selected depth. For example, in one non-limited application, the depth for image initialization may be 6 cm. Regardless of the particular mechanism for initialization or, if dept, the particular depth, at step 442, a cue is provided to the user. In one non-limiting example, the cure can communicate to the user to move caudally until bifurcation is detected. Then, at step 444, calibration is turned on. In one non-limiting example, the calibration can be turned on while cuing the user to move cranially. When the vessel(s) are detected at step 446, at step 448 the process finds the deepest vessel and calculates the buffer, for example, to the image bottom. At step 450, the buffer is set. In one non-limiting example, if an artery, the buffer may be set to 1.25 cm, else the buffer may be set to 0.75 cm. At step 452, adjustments may be made, for example, by rounding up to the nearest integer.

At step 454, the data is saved and at step 456, the data is sorted. For example, at step 454, non-zero depths may be saved in an array. Then, at step 456, the array is sorted, such that, at step 458, a threshold can be calculated based thereon. In one non-limiting example, the threshold may be at a selected percentile, such as the 75th percentile. Then at step 460, the image depth can be updated, for example, to the calculated depth. At step 462, the data can be cleared at the process repeated for the next set of detected vessels.

Thus, an automated gain control based on depth may be configured to balance too much gain that results in washout and artifacts, with too low of gain that results in a lack of signal. A machine learning or AI routine may be used to determine optimal image depth and gain such that the vessels of interest are well visualized. Since spatial resolution is poorer outside the ultrasound focal zone, the AI may automatically adjust the image depth so that the vessels are as close as possible to the center of the focal zone, while also ensuring the vessels are not cut off at the bottom of the image. The image gain optimization may be performed with histogram analysis of pixel intensities. The gain is adjusted to reach a dynamic range of intensities determined from well-gained training images.

Automatic gain control may start at a maximum depth, and the vessel detection model may be run. If a vessel is found, gain may be swept and an optimal gain may be found based on the optimal depth calculated for the vessel centroid. The ultrasound probe may then be reset to the optimal depth setting with the optimal gain, or gain may be swept if not at an optimal setting.

In some configurations, an integrated guidewire advancement may be used where a guidewire is included in the needle injection system. A rooter configuration may be used for containing and delivering the guidewire. The guidewire may expand into the inner diameter of the spool with an evenly distributed outward force. As the spool spins the guidewire may be extracted via a push force from the friction. As the guidewire navigates turns and tight spaces, there may be a net resistance force. As the resistance force increases, so will the outward force and consequently so will the friction, such that the friction will be greater than the resistance force, which allows for the friction force to push the guidewire as desired.

In some configurations, an integrated sheath and guidewire and deployment mechanism may be used. Using a shuttle, a sheath, needle, and guidewire may be selectively deployed into a subject as desired.

In some configurations, a safe method of cartridge-based guidewire and sheath insertion may be used that prevents sharps from being exposed outside of the system when the needle is not being inserted. The guidewire, sheath, or the system itself may be used without the needle tip ever being exposed as the needle is always fully enclosed in the cartridge when not being deployed. This provides for patient and operator inadvertent stick safety, reduces the likelihood of infection, and provides for increased speed of deployment.

In some configurations, stabilizing elements may be used to keep the device centered while scanning with ultrasound. In a non-limiting example, a cric attachment may be used where a tracheal guide keeps the device centered on the trachea midline. An ultrasound pad may be used as a standoff so that a cricothyroid membrane can be simultaneously imaged and inserted through.

Machine learning or AI algorithms may also be used to detect neck landmarks including but not limited to cricothyroid membrane, thyroid cartilage, thyroid glands, cricoid cartilage, infrahyoidmuscles (strap muscles), tracheal rings, and internal jugular veins in order to provide injection guidance for the needle. Image frames may be classified by the presence of one or more landmarks in the field of view and bounding box detection or segmentation may be used to localize the landmarks within the image.

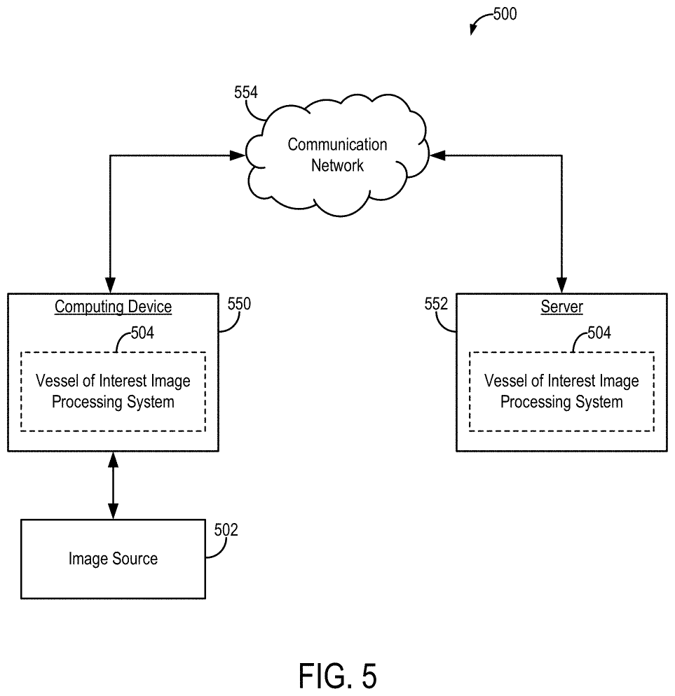

Referring to FIG. 5, an example of a system 500 for generating and implementing a hybrid machine learning and mechanistic model in accordance with some embodiments of the systems and methods described in the present disclosure is shown. As shown in FIG. 5, a computing device 550 can receive one or more types of data (e.g., ultrasound, multiparametric MRI data, vessel of interest image data, and the like) from image source 502. In some embodiments, computing device 550 can execute at least a portion of a vessel of interest image processing system 504 to generate images of a vessel of interest, or otherwise segment a vessel of interest from data received from the image source 502.

Additionally or alternatively, in some embodiments, the computing device 550 can communicate information about data received from the image source 502 to a server 552 over a communication network 554, which can execute at least a portion of the vessel of interest image processing system 504 to generate images of a vessel of interest, or otherwise segment a vessel of interest from data received from the image source 502. In such embodiments, the server 552 can return information to the computing device 550 (and/or any other suitable computing device) indicative of an output of the vessel of interest image processing system 504 to generate images of a vessel of interest, or otherwise segment a vessel of interest from data received from the image source 502.

In some embodiments, computing device 550 and/or server 552 can be any suitable computing device or combination of devices, such as a desktop computer, a laptop computer, a smartphone, a tablet computer, a wearable computer, a server computer, a virtual machine being executed by a physical computing device, and so on. The computing device 550 and/or server 552 can also reconstruct images from the data.

In some embodiments, image source 502 can be any suitable source of image data (e.g., measurement data, images reconstructed from measurement data), such as an ultrasound system, another computing device (e.g., a server storing image data), and so on. In some embodiments, image source 502 can be local to computing device 550. For example, image source 502 can be incorporated with computing device 550 (e.g., computing device 550 can be configured as part of a device for capturing, scanning, and/or storing images). As another example, image source 502 can be connected to computing device 550 by a cable, a direct wireless link, and so on. Additionally or alternatively, in some embodiments, image source 502 can be located locally and/or remotely from computing device 550, and can communicate data to computing device 550 (and/or server 552) via a communication network (e.g., communication network 554).

In some embodiments, communication network 554 can be any suitable communication network or combination of communication networks. For example, communication network 554 can include a Wi-Fi network (which can include one or more wireless routers, one or more switches, etc.), a peer-to-peer network (e.g., a Bluetooth network), a cellular network (e.g., a 3G network, a 4G network, etc., complying with any suitable standard, such as CDMA, GSM, LTE, LTE Advanced, WiMAX, etc.), a wired network, and so on. In some embodiments, communication network 108 can be a local area network, a wide area network, a public network (e.g., the Internet), a private or semi-private network (e.g., a corporate or university intranet), any other suitable type of network, or any suitable combination of networks. Communications links shown in FIG. 5 can each be any suitable communications link or combination of communications links, such as wired links, fiber optic links, Wi-Fi links, Bluetooth links, cellular links, and so on.

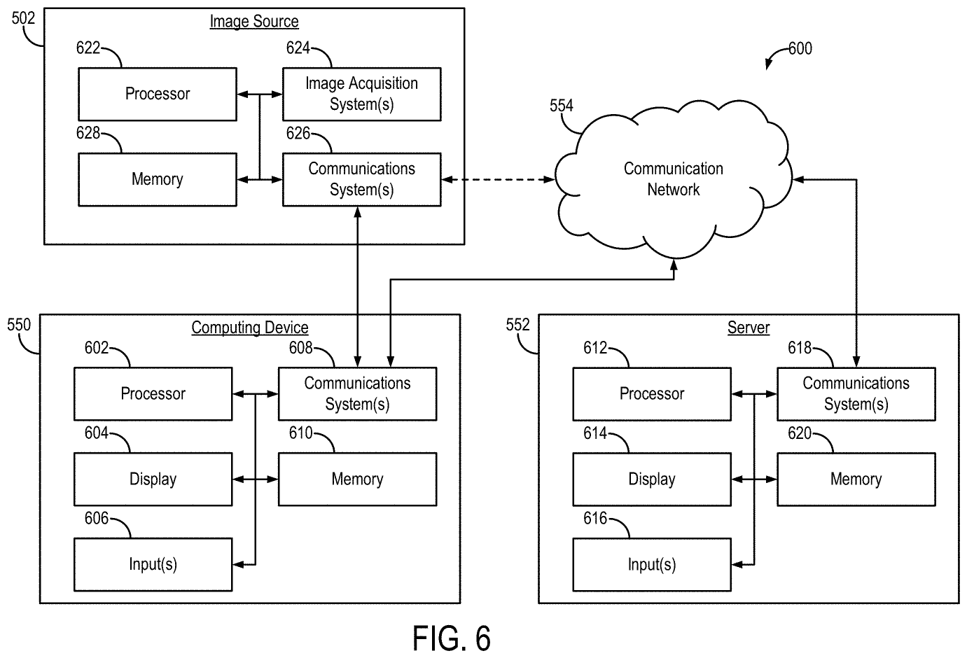

Referring now to FIG. 6, an example of hardware 600 that can be used to implement image source 502, computing device 550, and server 554 in accordance with some embodiments of the systems and methods described in the present disclosure is shown. As shown in FIG. 6, in some embodiments, computing device 550 can include a processor 602, a display 604, one or more inputs 606, one or more communication systems 608, and/or memory 610. In some embodiments, processor 602 can be any suitable hardware processor or combination of processors, such as a central processing unit (“CPU”), a graphics processing unit (“GPU”), and so on. In some embodiments, display 604 can include any suitable display devices, such as a computer monitor, a touchscreen, a television, and so on. In some embodiments, inputs 606 can include any suitable input devices and/or sensors that can be used to receive user input, such as a keyboard, a mouse, a touchscreen, a microphone, and so on.

In some embodiments, communications systems 608 can include any suitable hardware, firmware, and/or software for communicating information over communication network 554 and/or any other suitable communication networks. For example, communications systems 608 can include one or more transceivers, one or more communication chips and/or chip sets, and so on. In a more particular example, communications systems 608 can include hardware, firmware and/or software that can be used to establish a Wi-Fi connection, a Bluetooth connection, a cellular connection, an Ethernet connection, and so on.

In some embodiments, memory 610 can include any suitable storage device or devices that can be used to store instructions, values, data, or the like, that can be used, for example, by processor 602 to present content using display 604, to communicate with server 552 via communications system(s) 608, and so on. Memory 610 can include any suitable volatile memory, non-volatile memory, storage, or any suitable combination thereof. For example, memory 610 can include RAM, ROM, EEPROM, one or more flash drives, one or more hard disks, one or more solid state drives, one or more optical drives, and so on. In some embodiments, memory 610 can have encoded thereon, or otherwise stored therein, a computer program for controlling operation of computing device 550. In such embodiments, processor 602 can execute at least a portion of the computer program to present content (e.g., images, user interfaces, graphics, tables), receive content from server 552, transmit information to server 552, and so on.

In some embodiments, server 552 can include a processor 612, a display 614, one or more inputs 616, one or more communications systems 618, and/or memory 620. In some embodiments, processor 612 can be any suitable hardware processor or combination of processors, such as a CPU, a GPU, and so on. In some embodiments, display 614 can include any suitable display devices, such as a computer monitor, a touchscreen, a television, and so on. In some embodiments, inputs 616 can include any suitable input devices and/or sensors that can be used to receive user input, such as a keyboard, a mouse, a touchscreen, a microphone, and so on.

In some embodiments, communications systems 618 can include any suitable hardware, firmware, and/or software for communicating information over communication network 554 and/or any other suitable communication networks. For example, communications systems 618 can include one or more transceivers, one or more communication chips and/or chip sets, and so on. In a more particular example, communications systems 618 can include hardware, firmware and/or software that can be used to establish a Wi-Fi connection, a Bluetooth connection, a cellular connection, an Ethernet connection, and so on.

In some embodiments, memory 620 can include any suitable storage device or devices that can be used to store instructions, values, data, or the like, that can be used, for example, by processor 612 to present content using display 614, to communicate with one or more computing devices 550, and so on. Memory 620 can include any suitable volatile memory, non-volatile memory, storage, or any suitable combination thereof. For example, memory 620 can include RAM, ROM, EEPROM, one or more flash drives, one or more hard disks, one or more solid state drives, one or more optical drives, and so on. In some embodiments, memory 620 can have encoded thereon a server program for controlling operation of server 552. In such embodiments, processor 612 can execute at least a portion of the server program to transmit information and/or content (e.g., data, images, a user interface) to one or more computing devices 550, receive information and/or content from one or more computing devices 550, receive instructions from one or more devices (e.g., a personal computer, a laptop computer, a tablet computer, a smartphone), and so on.

In some embodiments, image source 502 can include a processor 622, one or more image acquisition systems 624, one or more communications systems 626, and/or memory 628. In some embodiments, processor 622 can be any suitable hardware processor or combination of processors, such as a CPU, a GPU, and so on. In some embodiments, the one or more image acquisition systems 624 are generally configured to acquire data, images, or both, and can include an RF transmission and reception subsystem of an MRI system. Additionally or alternatively, in some embodiments, one or more image acquisition systems 624 can include any suitable hardware, firmware, and/or software for coupling to and/or controlling operations of an MRI system or an RF subsystem of an MRI system. In some embodiments, one or more portions of the one or more image acquisition systems 624 can be removable and/or replaceable.

Note that, although not shown, image source 502 can include any suitable inputs and/or outputs. For example, image source 502 can include input devices and/or sensors that can be used to receive user input, such as a keyboard, a mouse, a touchscreen, a microphone, a trackpad, a trackball, and so on. As another example, image source 502 can include any suitable display devices, such as a computer monitor, a touchscreen, a television, etc., one or more speakers, and so on.

In some embodiments, communications systems 626 can include any suitable hardware, firmware, and/or software for communicating information to computing device 550 (and, in some embodiments, over communication network 554 and/or any other suitable communication networks). For example, communications systems 626 can include one or more transceivers, one or more communication chips and/or chip sets, and so on. In a more particular example, communications systems 626 can include hardware, firmware and/or software that can be used to establish a wired connection using any suitable port and/or communication standard (e.g., VGA, DVI video, USB, RS-232, etc.), Wi-Fi connection, a Bluetooth connection, a cellular connection, an Ethernet connection, and so on.

In some embodiments, memory 628 can include any suitable storage device or devices that can be used to store instructions, values, data, or the like, that can be used, for example, by processor 622 to control the one or more image acquisition systems 624, and/or receive data from the one or more image acquisition systems 624; to images from data; present content (e.g., images, a user interface) using a display; communicate with one or more computing devices 550; and so on. Memory 628 can include any suitable volatile memory, non-volatile memory, storage, or any suitable combination thereof. For example, memory 628 can include RAM, ROM, EEPROM, one or more flash drives, one or more hard disks, one or more solid state drives, one or more optical drives, and so on. In some embodiments, memory 628 can have encoded thereon, or otherwise stored therein, a program for controlling operation of image source 502. In such embodiments, processor 622 can execute at least a portion of the program to generate images, transmit information and/or content (e.g., data, images) to one or more computing devices 550, receive information and/or content from one or more computing devices 550, receive instructions from one or more devices (e.g., a personal computer, a laptop computer, a tablet computer, a smartphone, etc.), and so on.

In some embodiments, any suitable computer readable media can be used for storing instructions for performing the functions and/or processes described herein. For example, in some embodiments, computer readable media can be transitory or non-transitory. For example, non-transitory computer readable media can include media such as magnetic media (e.g., hard disks, floppy disks), optical media (e.g., compact discs, digital video discs, Blu-ray discs), semiconductor media (e.g., random access memory (“RAM”), flash memory, electrically programmable read only memory (“EPROM”), electrically erasable programmable read only memory (“EEPROM”)), any suitable media that is not fleeting or devoid of any semblance of permanence during transmission, and/or any suitable tangible media. As another example, transitory computer readable media can include signals on networks, in wires, conductors, optical fibers, circuits, or any suitable media that is fleeting and devoid of any semblance of permanence during transmission, and/or any suitable intangible media.

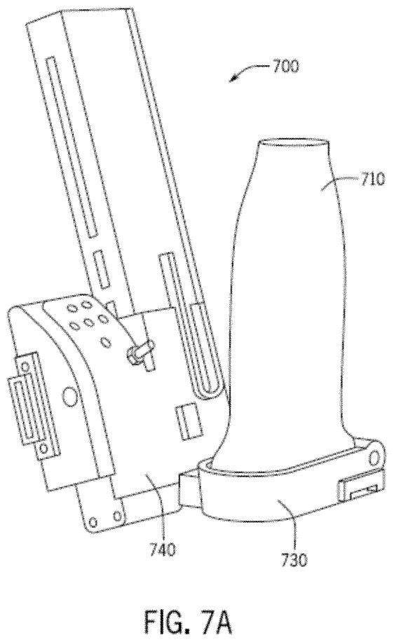

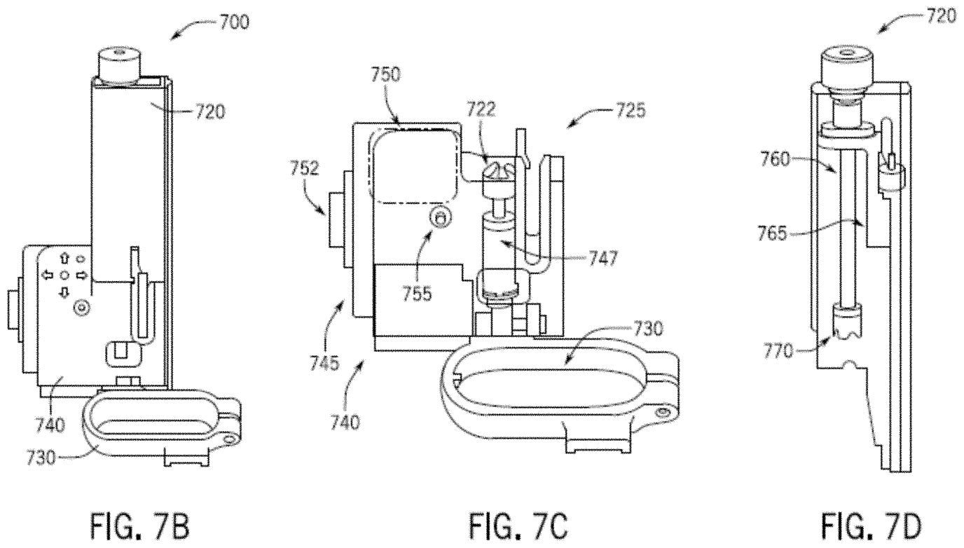

Referring to FIG. 7A, a perspective view of a non-limiting example interventional device guide injection assembly 700 coupled to an ultrasound probe 710 is shown. Base 740 is shown with ultrasound handle fixture 730 that provides detachable coupling to ultrasound probe 710. The injection assembly 700 may be attached to any ultrasound device, such as by being strapped onto an ultrasound probe 710 using the ultrasound handle fixture 730. Base 740 may include a mechanical support resting on the skin in order to minimize kick-back and improve needle insertion accuracy.

Referring to FIG. 7B, a side view of the interventional device guide injection assembly 700 of FIG. 7A is shown. In a non-limiting example, base 740 contains a motor to set the angle at which the interventional device, which may be a needle, will be inserted. The base 740 may also contain a second drive motor to drive the interventional device to the desired depth. The motor may be controlled to vary the needle insertion speed at different insertion depths, e.g., the needle may be inserted relatively slowly through the skin to minimize kick-back and improve accuracy, and then inserted faster subsequently. In some configurations, the drive motor function may be replaced or augmented by a spring or any suitable method of storing mechanical energy, and an additional motor or other suitable method of mechanical actuation to enable injection into a subject. Cartridge 720 is detachably coupled to base 740 and may be configured for the intervention being performed. In non-limiting examples, cartridge 720 may include configurations to treat indications requiring vascular access, tension pneumothorax, establishing of an airway, image guided tumor ablation or other image guided targeted cancer therapy, such as radiofrequency ablation, ethanol ablation, cryoablation, electroporation and the like, or percutaneous minimally invasive surgery, such as ligament release and the like. Non-limiting example cartridge configurations are listed in Table 1 below.

TABLE 1
Non-limiting example cartridge configurations

| | | Cartridge | |
|---|---|---|---|
| Intervention | | Generation | Capability |
| Vascular | Femoral | 1 | Needle only |
| | Artery/Vein | 2 | Needle with dilator and/or |
| | | | guide wire (or similarly |
| | | | functioning guide) |
| | | 3 | REBOA, clotting agent, |
| | | | other intervention |
| | Internal | 1 | Needle only |
| | Jugular | 2 | Needle with dilator and/or |
| | Vein | | guide wire (or similarly |
| | | | functioning guide) |
| | | 3 | REBOA, clotting agent, |
| | | | other intervention |
| Air | Cricothyrotomy | 1 | Needle only |
| | (or similar | 2 | Breathing tube |
| | methods of | 3 | Breathing tube + |
| | establishing | | forced air |
| | airway access) | | |
| | Tension | 1 | Needle only |
| | Pneumothorax | 2 | Chest tube |
| Abdomen | Ascites | 1 | Needle only |
| | | 2 | Catheter |
| | Bladder | 1 | Needle only |
| | Pregnant | 1 | Needle only |
| | uterus | | |
| | amniocentesis | | |
| Soft | Focal | 1 | Needle only |
| tissue | lesion/tumor | | |
| | biopsy | | |
| Image | Focal anatomy/ | 1 | Needle with dilator and/or |
| Guided | lesion/tumor | | guide wire (or similarly |
| Tumor | | | functioning guide) and an |
| Ablation | | | ablation device |

Referring to FIG. 7C is a side view of the base and ultrasound probe fixture for the interventional device guide of FIG. 7B. Base 740 includes a drive motor 745 to set an insertion angle and/or depth for an interventional device held by cartridge slot 725 coupled by cartridge coupling 722. Advancement motor 747 may be included to advance an interventional device with activation by advancement control 755, which in a non-limiting example is a button. Electrical interface connector 752 may provide communication to an ultrasound imaging system or separate display system. User guidance signal 750 provide feedback to a user and may take the form or any display intending to direct the user in gross and/or precise placement of the device. In a non-limiting example, user guidance signal 750 includes an arrangement of LEDs. In some configurations, user guidance signal 750 may be coupled to the cartridge 720 and may be specific to the particular indication being treated.

Referring to FIG. 7D is a cross-section of a non-limiting example cartridge 720 compatible with the injection assembly 700 of FIG. 7B. Lead screw 760 may provide for actuation of base coupling 770 to couple the non-limiting example cartridge 720 to base 740 in FIG. 7B. Needle carriage 765 is shown as a non-limiting example of a needle cartridge application.

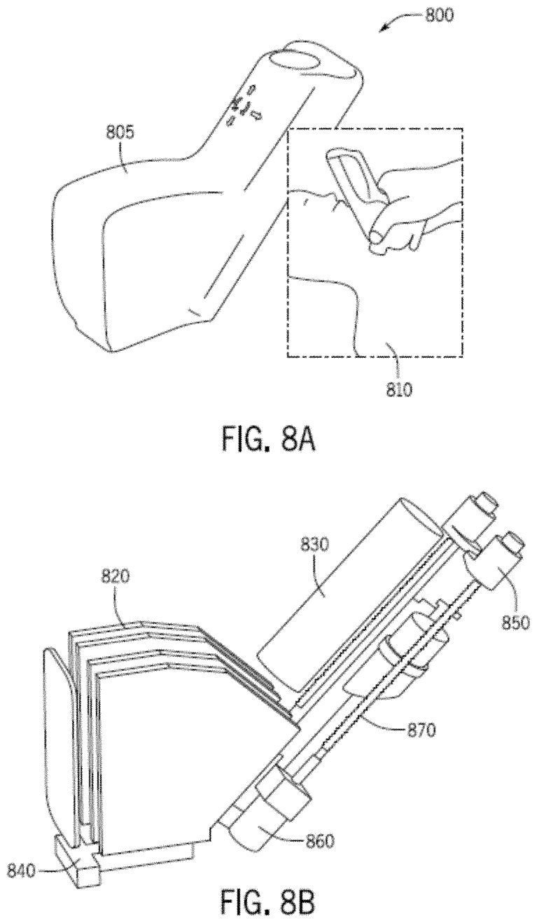

Referring to FIG. 8A, a perspective view of a non-limiting example interventional device guide integrated with an ultrasound probe is shown. Integrated interventional device guide 800 is shown being placed on a subject 810. The integrated interventional device guide 800 may include functionality similar to injection assembly 700 described above with integration with an ultrasound probe. The integrated interventional device guide 800 may be ultrasound guided, and may employ machine learning or artificial intelligence for identifying a target structure for penetration and guiding penetration of the target structure, in accordance with the present disclosure. The integrated ultrasound transducer may provide for excitation, for reading a source, for processing ultrasound signals, and the like. Integrated interventional device guide 800, may include onboard artificial intelligence algorithms, motors, associated drive circuitry, other electronics/mechanics, and the like fit within a housing 805 for the integrated device guide 800. A cartridge, such as described herein, may be detachably coupled to integrated interventional device guide 800. In some configurations, the integrated interventional device guide 800 may be robotically controlled.

Referring to FIG. 8B is an exploded view of the integrated interventional device guide 800 and ultrasound probe of FIG. 8A is shown. Circuit boards 820 may provide for ultrasound guidance from ultrasound transducers 840, and may employ machine learning or artificial intelligence for identifying a target structure for penetration and guiding penetration of the target structure, in accordance with the present disclosure. Battery 830 may provide power for the integrated device. One battery cell is shown in FIG. 8B, but it is to be appreciated that any number of battery cells may be used, such as two cells for extended life, or any other form of power supply. Drivetrain 850 may provide for independent needle or interventional device insertion and cannula insertion. Needle and cannula 870 may be inserted into a subject with motors 860.

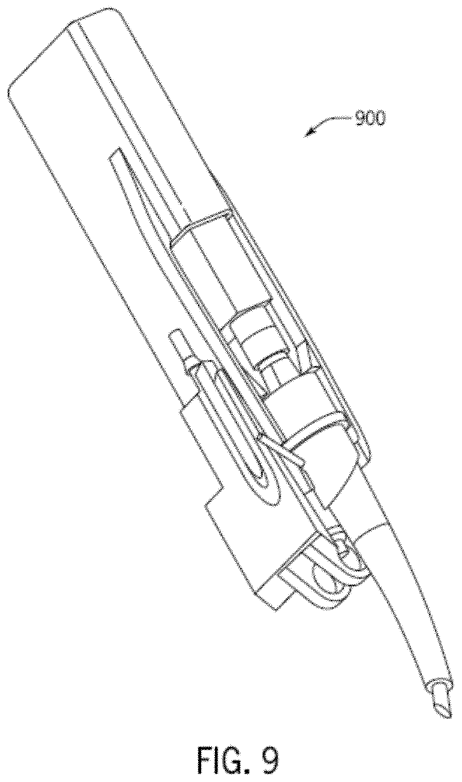

Referring to FIG. 9 is perspective view of a non-limiting example cricothyrotomy cartridge 900 for use in accordance with the present disclosure. As indicated in Table 1 above, different clinical indications may require different types of needles or other hardware/drugs to be introduced into the body. For example, options may include one of a needle, wire, dilator, breathing tube, chest tube, vascular catheter, blood clotting agent, a drainage catheter, an injectable delivery carrier, such as a hydrogel, or drug. In a non-limiting example, in the case of non-compressible hemorrhage, blood products may need to be rapidly introduced and a needle sheath may provide a path of adequate diameter for rapid introduction of fluid. In another non-limiting example, a catheter may need to be introduced, or a dilating element with larger lumen may be required. Each cartridge may be designed, and clearly labeled with, an intended application. In some configurations, the system may be capable of knowing which type of cartridge device is “plugged” into it. This information may be conveyed through electrical communication between the cartridge and the base, such as radio frequency or direct conducted signals, or through optical communication between the cartridge and the base, or through a mechanical keying specific to the cartridge/base assembly that indicates the cartridge type used, and the like. In a non-limiting example of a mechanical keying, the Femoral Artery/Vein Generation 1 cartridge of Table 1 could be configured such that it depresses a first button in the cartridge slot in the base, whereas the generation 2 cartridge in this family could be configured to depress a second button. In this manner, the base may distinguish between which cartridges have been inserted. In some configurations, the cartridge may be inside of the sterile surgical barrier with the base external the sterile barrier, such that communication of the cartridge type may be performed through the barrier to ensure safe, effective treatment.

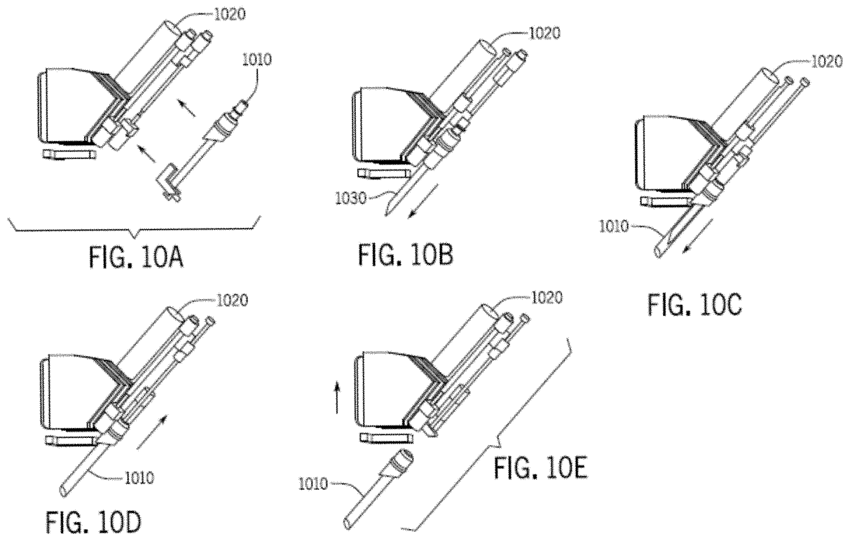

Referring to FIGS. 10A-E, side views of inserting and removing a non-limiting example dilating component into a subject is shown. Some types of cartridges shown in Table 1 may require more than a single step needle insertion process. In a non-limiting example, a cartridge may be configured to install a dilated lumen, which may include a multi-step process. In a non-limiting example, installing a breathing tube through the cricothyroid membrane may include a coaxial assembly consisting of a sharp center element for puncturing and initial path guidance in addition to a coaxial element for dilation and eventual passage of air, which may be introduced according to FIGS. 10A-E.

The sequence shown in FIGS. 10A-10E may be entirely automated by the motors or other mechanical actuation in the system, or may be a combination of automated actuation and human handling. Referring to FIG. 10A, a side views of inserting a non-limiting example dilating component 1010 into a subject is shown. In some configurations, a protector may be removed to insert a disposable version of the dilator 1010 to maintain sterility and safety.

Referring to FIG. 10B a side view of aligning a non-limiting example dilating component 1010 with the interventional device guide 1020 is shown. Needle 1030 may be deployed after device alignment, which may be coaxial with dilating component 1010. In some configurations, the receiving anatomy may be more sensitive to damage or additional mechanical guidance may be required for proper introduction of the larger diameter element. In such configurations, a “guide-wire” device may be used to temporarily protrude from the tip of the inserted assembly, in a function similar to that of the guide-wire used in the Seldinger technique. The “guide-wire” device may be deployed between steps depicted in FIG. 10B and FIG. 10C.

Referring to FIG. 10C, a side view of advancing a non-limiting example dilating component 1010 into the subject is shown. Dilating component 1010 may be advanced over, and may be coaxial with, needle 1030. Dilating component 1010 may provide for expanded access into the subject after insertion. Referring to FIG. 10D, a side view of retracting the needle 1030 from the subject is shown. Referring to FIG. 10E, a side view of removing the interventional device guide 1020 is show where dilating component 1010 is retained in the subject and may be used for access from an interventional device.

The present disclosure has described one or more preferred embodiments, and it should be appreciated that many equivalents, alternatives, variations, and modifications, aside from those expressly stated, are possible and within the scope of the invention.

1. A system for guiding an interventional device in an interventional procedure of a subject, comprising:
an ultrasound probe;
a guide system coupled to the ultrasound probe and configured to guide the interventional device into a field of view (FOV) of the ultrasound probe;
a non-transitory memory having instructions stored thereon;
a processor configured to access the non-transitory memory and execute the instructions, wherein the processor is caused to:
access image data acquired from the subject using the ultrasound probe, wherein the image data include at least one image of a target structure of the subject and surrounding structures;
determine, from the image data, a location of the target structure within the subject and at least one critical structure to avoid; and
determine a safe pathway for the interventional device to reach the target structure without impinging on the at least one critical structure based upon the location of the target structure and the at least one critical structure.

2. The system of claim 1, wherein the processor is further caused to determine an insertion angle for the interventional device using at least one of an insertion point location, the location of the target structure, or the location of the at least one critical structure.

3. The system of claim 2, wherein the processor is further caused to determine a distance to a distal wall of the target structure from the insertion point location.

4. The system of claim 3, wherein the processor is further caused to determine an overshoot estimation based upon the determined insertion angle and the determined distance to the distal wall of the target structure.

5. The system of claim 1, wherein the processor is further caused to vibrate the interventional device.

6. The system of claim 1, wherein the processor is further caused to reduce an insertion speed of the interventional device as the interventional device approaches the target structure.

7. The system of claim 1, wherein the target structure is one of an artery, a vein, a femoral artery, a femoral vein, a jugular vein, a peripheral vein, a subclavian vein, an airway, a lumen, a luminal organ, a body cavity, a fluid filled anatomic space, a location requiring biopsy, a breast, a kidney, a lymph node, a spinal canal, a location requiring nerve block, a peritoneal space or a pleural space.

8. The system of claim 1, wherein the processor is configured to receive a plurality of images of the target structure of the subject acquired in real time to access the image data.

9. The system of claim 8, wherein the plurality of images include a plurality of views of the target structure, and wherein the processor is configured to assess the plurality of views to identify a critical structure in the subject and identify a location on the subject where the interventional device reaches the target structure from an insertion point location without penetrating the critical structure in the subject.

10. The system of claim 9, wherein the critical structure includes at least one of a bone, a lung, heart, an unintended blood vessel, a non-target organ, or a nerve.

11. The system of claim 1, wherein the processor is further caused to determine a blood flashback.

12. The system of claim 11, wherein the processor is further caused to advance the interventional device toward the critical structure in the absence of blood flashback.

13. The system of claim 1, wherein the processor is further caused to determine an overshoot estimation and guide the interventional device to penetrate the target structure without penetrating a distal wall of the target structure based upon the overshoot estimation.

14. The system of claim 1, wherein the guide system includes a removable cartridge coupled to a base of the guide system, wherein the cartridge contains the interventional device.

15. The system of claim 14, wherein the interventional device is one of a needle, wire, dilator, breathing tube, chest tube, vascular catheter, blood clotting agent, a drainage catheter, an injectable delivery carrier, a hydrogel, or drug.

16. The system of claim 15, wherein the interventional device is configured to provide at least one of vascular access, access to an organ or body cavity, perform cricothyrotomy, take a tissue sample, alleviate pneumothorax, drain fluid from a body cavity, drain pus from an abscess, or drain cerebrospinal fluid from a spinal canal.

17. The system of claim 1 further comprising a guidewire configured to be delivered with the interventional device to the target structure.

18. A system for guiding an interventional device in an interventional procedure of a subject, comprising:
an ultrasound probe;
a guide system coupled to the ultrasound probe and configured to guide the interventional device into a field of view (FOV) of the ultrasound probe;
a non-transitory memory having instructions stored thereon;
a processor configured to access the non-transitory memory and execute the instructions, wherein the processor is caused to:
access image data acquired from the subject using the ultrasound probe, wherein the image data include at least one image of a target structure of the subject;
determine, from the image data, a cross section of the target structure within the subject; and
fit a shape for the cross section of the target structure to determine a centroid for the target structure and guide the interventional device to the centroid to penetrate the target structure.

19. The system of claim 18, wherein the shape is an ellipse.

20. The system of claim 18, wherein the processor is further caused to determine an overshoot estimation for guiding the interventional device to penetrate the target structure without penetrating a distal wall of the target structure based upon the overshoot estimation.

21. The system of claim 18, wherein the processor is further caused to determine an Otsu threshold for the cross section of the target structure.

22. The system of claim 21, wherein the processor is further caused to determine an erosion for the cross section of the target structure, and a dilation for the erosion to determine a wall for the target structure.

23. The system of claim 22, wherein the processor is further caused to generate a plurality of spokes for the eroded and dilated cross section of the target structure for fitting the ellipse.

24. The system of claim 18, wherein the processor is further caused to vibrate the interventional device.

25. The system of claim 18, wherein the processor is further caused to reduce an insertion speed of the interventional device as the interventional device approaches the target structure.

26. The system of claim 18, wherein the target structure is one of an artery, a vein, a femoral artery, a femoral vein, a jugular vein, a peripheral vein, a subclavian vein, an airway, a lumen, a luminal organ, a body cavity, a fluid filled anatomic space, a location requiring biopsy, a breast, a kidney, a lymph node, a spinal canal, a location requiring nerve block, a peritoneal space a pleural space, abscess, amniotic sac, umbilical vessel, or a nerve.

27. The system of claim 18, wherein the processor is configured to receive a plurality of images of the target structure of the subject acquired in real time to access the image data.

28. The system of claim 27, wherein the plurality of images include a plurality of views of the target structure, and wherein the processor is configured to assess the plurality of views to identify a critical structure in the subject and identify a location on the subject where the interventional device reaches the target structure from an insertion point location without penetrating the critical structure in the subject.

29. The system of claim 28, wherein the critical structure includes at least one of a bone, an unintended blood vessel, a non-target organ, or a nerve.

30. The system of claim 18, wherein the processor is further caused to determine a blood flashback.

31. The system of claim 18, wherein the guide system includes a removable cartridge coupled to a base of the guide system, wherein the cartridge contains the interventional device.

32. The system of claim 31, wherein the interventional device is one of a needle, wire, dilator, breathing tube, chest tube, vascular catheter, blood clotting agent, a drainage catheter, an injectable delivery carrier, a hydrogel, or drug.

33. The system of claim 32, wherein the interventional device is configured to provide at least one of vascular access, access to an organ or body cavity, perform cricothyrotomy, take a tissue sample, or alleviate pneumothorax.

34. The system of claim 18, wherein the guide system, non-transitory memory, or processor form a modular apparatus configured to be coupled to the ultrasound probe or other devices to carry out a desired medical procedure.

35. A method of controlling a robotically controlled system to determine a safe pathway for guiding an interventional device in an interventional procedure of a subject, the method including causing a processor to carry out steps comprising:
access image data acquired from the subject using an ultrasound probe, wherein the image data include at least one image of a target structure of the subject and at least one critical structure within the subject;
determine, from the image data, a location of the target structure within the subject and the at least one critical structure; and
determine a safe pathway for the interventional device to reach the target structure without impinging on the at least one critical structure based upon the location of the target structure and the at least one critical structure.

36. The method of claim 35, wherein the processor is further caused to determine an insertion angle for the interventional device using at least one of an insertion point location, the location of the target structure, or the location of the at least one critical structure.

37. The method of claim 36, wherein the processor is further caused to determine a distance to a distal wall of the target structure from the insertion point location.

38. The method of claim 37, wherein the processor is further caused to determine an overshoot estimation based upon the determined insertion angle and the determined distance to the distal wall of the target structure.

39. The method of claim 1, wherein the processor is further caused to reduce an insertion speed of the interventional device as the interventional device approaches the target structure.

40. The method of claim 35, wherein the target structure is one of an artery, a vein, a femoral artery, a femoral vein, a jugular vein, a peripheral vein, a subclavian vein, an airway, a lumen, a luminal organ, a body cavity, a fluid filled anatomic space, a location requiring biopsy, a breast, a kidney, a lymph node, a spinal canal, a location requiring nerve block, a peritoneal space or a pleural space.

41. The method of claim 35, wherein the image data includes a plurality of views of the target structure, and wherein the processor is configured to assess the plurality of views to identify a critical structure in the subject and identify a location on the subject where the interventional device reaches the target structure from an insertion point location without penetrating the critical structure in the subject.

42. The method of claim 41, wherein the critical structure includes at least one of a bone, an unintended blood vessel, a non-target organ, or a nerve.

43. The method of claim 35, wherein the processor is further caused to determine a blood flashback.

44. The method of claim 43, wherein the processor is further caused to advance the interventional device toward the critical structure in the absence of blood flashback.

45. The method of claim 35, wherein the processor is further caused to determine an overshoot estimation and guide the interventional device to penetrate the target structure without penetrating a distal wall of the target structure based upon the overshoot estimation.

46. The method of claim 35, wherein the interventional device is one of a needle, wire, dilator, breathing tube, chest tube, vascular catheter, blood clotting agent, a drainage catheter, an injectable delivery carrier, a hydrogel, or drug, or is configured to provide at least one of vascular access, access to an organ or body cavity, perform cricothyrotomy, take a tissue sample, alleviate pneumothorax, drain fluid from a body cavity, drain pus from an abscess, or drain cerebrospinal fluid from a spinal canal.
